(19) United States
(12) Patent Application Publication
(10) Pub. No.: US 2026/0207044 A1
(43) Pub. Date: Jul. 23, 2026

(54) Systems and Methods for Using an Arthroscopic Assembly for Displaying and Selecting User Options and Parameters

(71) Applicants: Smith & Nephew, Inc., Memphis, TN (US); Smith & Nephew Asia Pacific Pte. Limited, Singapore (SG)

(72) Inventor: Nicholas Ryan LABRIOLA, Medway, MA (US)

(73) Assignees: Smith & Nephew, Inc., Memphis, TN (US); Smith & Nephew Asia Pacific Pte. Limited, Singapore (SG)

(21) Appl. No.: 19/451,118

(22) Filed: Jan. 16, 2026

Related U.S. Application Data
(60) Provisional application No. 63/746,368, filed on Jan. 17, 2025.

(51) Int. Cl. A61B 1/317; A61B 1/00

(52) U.S. Cl. CPC A61B 1/317; A61B 1/00045; A61B 1/00131; A61B 1/00183

(57) ABSTRACT

A surgical system may include: an arthroscope; a camera having a field of view via the arthroscope, the arthroscope being rotationally adjustable about an axis with respect to the camera; an indicia for tracking, in the field of view of the camera, the rotational position of the arthroscope about the axis; a display device; and one or more processor for: receiving video frames from the camera; displaying the video frames on the display device; determining a current location of the indicia in the video frames; displaying, on the display device, a field of user options in association with the video frames; based on the current location of the indicia in the video frames, automatically determining a corresponding location within the field of user options; determining a value associated with the corresponding location within the field of user options; and at least displaying the value on the display device.

### CROSS-REFERENCE

This application claims priority to United States Provisional Patent Application Serial No. 63/746,368 filed on January 17, 2025 and entitled "Leveraging Field Stop Mask Fiducial for Intraoperative Parameter Selection". The contents of the provisional patent application are incorporated herein by reference.

### BACKGROUND

An arthroscopic camera generally provides a surgeon with a window into a surgical site, such as the joint of a patient. For example, an arthroscopic camera may be used to provide a window into a knee joint in order to visualize and, along with surgical instruments inserted via minimally invasive portals, address injuries to the ligaments and/or bones of the knee joint.

Various technologies to process images captured by an arthroscopic camera to generate and provide information to a surgeon about the surgical site are available. For example, a three-dimensional bone model of a joint generated using cross-sectional images such as those created using MRI (Magnetic Resonance Imaging) and/or CT (Computed Tomography) may be registered in position and orientation to the actual bones by a surgeon while viewing the actual bones using an arthroscopic camera. Once registered, the three-dimensional bone models may be presented on a display screen according to how the surgeon is positioning and orienting the arthroscopic camera, and may thus be useful for computer-assisted surgical navigation.

Registration of a three-dimensional bone model to an actual bone may be aided by attaching a bone fiducial to a portion of the actual bone in the surgical site using a fastener such as a screw, and keeping the bone fiducial in the field of view of the arthroscopic camera while "painting" portions of the bone exposed to view with a separate touch probe. In this way, points on the bone may be associated with points on the three- dimensional model. Because the bone fiducial does not move with respect to the bone, it can be used as a positional anchor such that images from the arthroscopic camera can be processed to first locate and orient the bone fiducial and then to determine the relative position of a point being contacted by the touch probe. When enough points between the actual bone and the three-dimensional bone model are matched, the entire three- dimensional bone model can be positioned and oriented correctly with respect to the actual bone as long as the bone fiducial itself remains within the field of view of the arthroscopic camera. A number of systems, methods and procedures for conducting such registration are described in PCT Publication No. WO/2023/034194 to Quist et al. ("Quist").

It may be useful for a surgeon to use an arthroscopic camera to conduct other procedures such as taking measurements within a surgical site. For example, a surgeon may wish to measure a focal defect or the length of a tear, or to guide anatomic placement using measured values. Such other procedures are not typically conducted with the aid of computer-based image processing. For example, a surgeon may physically place a rigid ruler into the surgical site and, by visually studying the images produced by the arthroscopic camera while it captures the ruler within the surgical site, manually measure a particular portion of the anatomy. It would be useful if a surgeon could be aided in making such measurements or conducting other similar operations by the kinds of imaging processing that is employed for computer-assisted surgical navigation.

It is important in surgery to limit deviations from a planned workflow. If a surgeon has a planned workflow and, during surgery, wishes to make a quick unplanned measurement of, or otherwise characterize, something within the surgical site, the surgeon may have to "break scrub" to interact with a tablet or other device in order to record the measurement or to otherwise select options or parameters during the planned workflow.

Improvements for efficiently and accurately using image processing to display and enable a user to select user options without requiring deviations from planned workflows or unduly requiring a user to "break scrub" are therefore desirable.

### SUMMARY

One example is a surgical system. The surgical system may comprise an arthroscope; a camera attached to the arthroscope and having a field of view via the arthroscope, wherein the arthroscope is rotationally adjustable about an axis with respect to the camera; an indicia associated with the arthroscope for tracking, in the field of view of the camera, the rotational position of the arthroscope about the axis; a display device; and one or more processor for: receiving video frames from the camera; displaying the video frames on the display device; and during displaying the video frames on the display device: determining a current location of the indicia in the video frames; displaying, on the display device, a field of user options in association with the video frames; based on the current location of the indicia in the video frames, automatically determining a corresponding location within the field of user options; determining a value associated with the corresponding location within the field of user options; and displaying the value on the display device.

In examples, the field of user options may be a range of numbers.

In examples, the corresponding location within the range of numbers may a particular number within the range of numbers, and determining the value associated with the corresponding location may comprise determining the value to be the particular number.

In examples, the corresponding location within the range of numbers may be a location between two particular numbers within the range of numbers, and determining the value associated with the corresponding location may comprise determining the value to be a number that is between the two particular numbers.

In examples, the field of user options may be a set of menu options.

In examples, the set of menu options may comprise two menu options.

In examples, the one or more processor may be for: receiving a user input signal to select the determined value; and conducting processing using the determined value.

In examples, the one or more processor may be for: receiving a user input signal to select the determined value; and conducting processing using the determined value.

In examples, conducting processing using the determined value may comprise storing the determined value.

In examples, the field of user options may be displayed on the display device responsive to receipt, by the one or more processor, of a user request.

In examples, a position on the display device at which the field of user options is displayed may be based on the current location of the indicia in the video frames at the time of the receiving of the user request.

Yet another example is a processor-implemented method. The method may comprise: receiving, by one or more processor, video frames from a camera attached to an arthroscope, wherein the camera has a field of view via the arthroscope and the arthroscope is rotationally adjustable about an axis with respect to the camera, wherein an indicia is associated with arthroscope for tracking, in the field of view of the camera, the rotational position of the arthroscope about the axis; displaying, by the one or more processor, the video frames on a display device; during displaying the video frames on the display device: determining, by the one or more processor, a current location of the indicia in the video frames; displaying, by the one or more processor, a field of user options in association with the video frames on the display device; based on the current location of the indicia in the video frames, automatically determining, by the one or more processor, a corresponding location within the field of user options; determining, by the one or more processor, a value associated with the corresponding location within the field of user options; and displaying, by the one or more processor, the value on the display device.

In examples, the field of user options may be a range of numbers.

In examples, the corresponding location within the range of numbers may be a particular number within the range of numbers, and determining the value associated with the corresponding location may comprise determining the value to be the particular number.

In examples, the corresponding location within the range of numbers may be a location between two particular numbers within the range of numbers, and determining the value associated with the corresponding location may comprise determining the value to be a number that is between the two particular numbers.

In examples, the field of user options may be a set of menu options.

In examples, the set of menu options may comprise two menu options.

In examples, the method may further comprise: receiving, by the one or more processor, a user input signal to select the determined value; and conducting processing, by the one or more processor, using the determined value.

In examples, conducting processing using the determined value may comprise storing the determined value.

In examples, the field of user options may be displayed on the display device responsive to receiving, by the one or more processor, a user request.

In examples, a position on the display device at which the field of user options is displayed may be based on the current location of the indicia in the video frames at a time of the receiving of the user request.

Other examples are provided in the following description and the accompanying drawings.

### BRIEF DESCRIPTION OF THE DRAWINGS

For a detailed description of example embodiments, reference will now be made to the accompanying drawings in which:

FIG. 1 shows a surgical system in accordance with at least some embodiments;

FIG. 2 shows a conceptual drawing of a surgical site with various objects within the surgical site tracked, in accordance with at least some embodiments;

FIG. 3 is an example video display of video frames as captured in the field of view of an arthroscopic camera showing portions of a femur and having visible therein a bone fiducial, in accordance with at least some embodiments;

FIG. 4A shows a side view of an arthroscopic assembly with an arthroscope in a first rotational position about a longitudinal central axis of the arthroscope with respect to an attached camera, in accordance with at least some embodiments;

FIG. 4B shows an example video display showing a first location of an indicia in the field of view of the attached camera corresponding to the rotational position of the arthroscope in FIG. 4A, and showing a range of selectable values and a current selectable value corresponding to the first location of the indicia;

FIG. 5A shows a side view of the arthroscopic assembly with the arthroscope in a second rotational position about the longitudinal central axis of the arthroscope with respect to the attached camera, in accordance with at least some embodiments;

FIG. 5B shows an example video display showing a second location of an indicia in the field of view of the attached camera corresponding to the rotational position of the arthroscope in FIG. 5A, and showing a range of selectable values and a current selectable value corresponding to the second location of the indicia;

FIG. 6A shows a mask that can be applied to the arthroscope to provide an indicia within the field of view of the attached camera that can track, in the field of view of the camera, the rotational position of the arthroscope about the axis;

FIG. 6B shows a field of view of an arthroscopic camera without the mask of FIG. 6A;

FIG. 6C shows the field of view of the arthroscopic camera including the mask of FIG. 6A having been applied to the arthroscope to provide the tracking indicia between the camera and the surgical site;

FIG. 7A shows an example video display showing a first location of an indicia in the field of view of an attached camera corresponding to the current rotational position of the arthroscope, and showing a menu of two selectable options and a current one of the selectable options corresponding to a current location of the indicia;

FIG. 7B shows the example video display of FIG. 7A in which the indicia is in a second location in the field of view of an attached camera corresponding to the current rotational position of the arthroscope as adjusted compared to FIG. 7A, and showing the menu of the two selectable options and a current one of the selectable options corresponding to a current location of the indicia;

FIG. 7C shows the example video display of FIGS. 7A and 7B after a user input signal to select a current selectable option has been received by the surgical system;

FIG. 8A shows an example video display showing a first location of an indicia in the field of view of an attached camera corresponding to the current rotational position of the arthroscope, and showing a menu of four selectable options and a current one of the selectable options corresponding to a current location of the indicia;

FIG. 8B shows the example video display of FIG. 8A in which the indicia is in a second location in the field of view of an attached camera corresponding to the current rotational position of the arthroscope as adjusted compared to FIG. 8A, and showing the menu of the four selectable options and a current one of the selectable options corresponding to a current location of the indicia;

FIG. 8C shows the example video display of FIGS. 8A and 7B 8B after a user input signal to select a current selectable option has been received by the surgical system;

FIG. 9 shows a method in accordance with at least some embodiments; and

FIG. 10 shows a computer system in accordance with at least some embodiments.

### DEFINITIONS

Various terms are used to refer to particular system components. Different companies may refer to a component by different names - this document does not intend to distinguish between components that differ in name but not function. In the following discussion and in the claims, the terms "including" and "comprising" are used in an open- ended fashion, and thus should be interpreted to mean "including, but not limited to...." Also, the term "couple" or "couples" is intended to mean either an indirect or direct connection. Thus, if a first device couples to a second device, that connection may be through a direct connection or through an indirect connection via other devices and connections.

"Processing structure" or "processor" shall mean a single processing device, processor, microprocessing device, microprocessor, computing device, computer, computer system or other device that, like these, can be instructed to and/or configured to conduct computational processing, or an arrangement of multiple processing devices, processors, microprocessing devices, microprocessors, computing devices, computers, computer systems and/or other devices that, like these, can be instructed to and/or configured to conduct computational processing.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various examples are directed to methods and systems for using an indicia associated with an arthroscope for tracking, in the field of view of an arthroscopic camera, the rotational position of the arthroscope about an axis with respect to the arthroscopic camera and using the tracking to enable a user to specify and select from options or parameters in fields of user options or parameters.

The techniques described herein are applicable to any type of arthroscopic work, whether measurement, anatomy characterization, ligament repair, or other arthroscopic work that is either planned as part of a workflow, or is a deviation from a planned workflow.

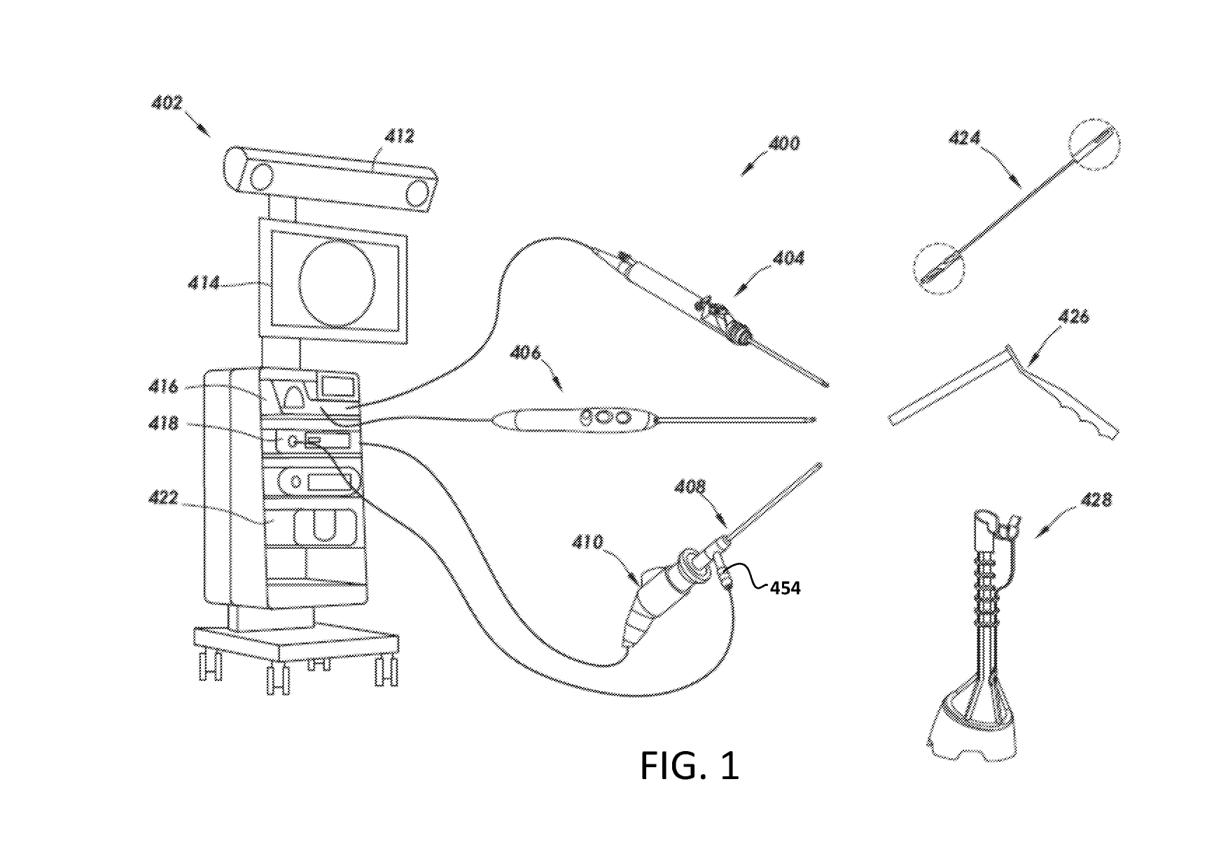

FIG. 1 shows a surgical system (not to scale) in accordance with at least some embodiments. In particular, the example surgical system 400 comprises a tower or device cart 402, an example mechanical resection instrument 404, an example plasma- based ablation instrument (hereafter just ablation instrument 406), and an arthroscopic assembly in the example form of an arthroscope 408 and attached camera head (or, simply, camera 410). The arthroscopic assembly defines a light connection or light post 454 to which light is provided, and the light is routed internally within the arthroscope 408 to illuminate a surgical field at the distal end of the arthroscope 708. The device cart 402 may comprise a camera 412 (illustratively shown as a stereoscopic camera), a display device 414, a resection controller 416, and a camera control unit (CCU) together with an arthroscopic light source and video controller 418. In example cases the CCU and video controller 418 provides light to the light post 454 of the arthroscope 408, and displays images such as video frames received from the camera 410. In example cases, the CCU and video controller 418 also implement various additional aspects, such as calibration of the arthroscopic assembly, calculating and/or displaying information for computer- guided surgical assistance, and/or other aspects. Thus, the CCU and video controller is hereafter referred to as surgical controller 418. In other cases, however, the CCU and video controller may be a separate and distinct system from the controller that handles various aspects, yet the separate devices would nevertheless be operationally coupled.

The example device cart 402 further includes a pump controller 422 (e.g., single or dual peristaltic pump). Fluidic connections of the mechanical resection instrument 404 and ablation instrument 406 are not shown so as not to unduly complicate the figure. Similarly, fluidic connections between the pump controller 422 and the patient are not shown so as not to unduly complicate the figure. In the example system, both the mechanical resection instrument 404 and the ablation instrument 406 are coupled to the resection controller 416 being a dual-function controller. In other cases, however, there may be a mechanical resection controller separate and distinct from an ablation controller. The example devices and controllers associated with the device cart 402 are merely examples, and other examples include vacuum pumps, patient-positioning systems, robotic arms holding various instruments, ultrasonic cutting devices and related controllers, patient-positioning controllers, and robotic surgical systems.

FIG. 1 further shows additional instruments that may be present during an example ACL repair. In particular, FIG. 1 shows an example guide wire or drill wire 424 and an aimer 426. The drill wire 424 may be used to create an initial or pilot tunnel through the bone requiring formation or repair of a bone tunnel. The example drill wire 424 is shown with magnified portions on each end, one to show the cutting elements on the distal end of the drill wire 424, and another magnified portion to show a connector for coupling to chuck of a drill. Once the surgeon drills the pilot tunnel, the surgeon and/or the surgical controller 418 may then assess whether the pilot tunnel matches or closes matches a planned-tunnel path. If the pilot tunnel is deemed sufficient, then the drill wire 424 may be used as a guide for creating the full-diameter throughbore for the tunnel, and possibly also for creating a counterbore associated with intercondylar notch to accommodate the graft. While in some cases the drill wire alone may be used when creating the pilot tunnel, in yet still other cases the surgeon may use the aimer 426 to help guide and place the drill wire 424 at the designed tunnel-entry location.

FIG. 1 also shows that the example system may comprise a calibration assembly 428. As explained in further detail in PCT Publication No. WO/2023/034194 to Quist et al. ("Quist"), the calibration assembly 428 may be used to detect optical distortion in images received by the surgical controller 418 through the arthroscope 408 and attached camera 410. Additional tools and instruments will be present, such as a drill for drilling with the drill wire 424, various reamers for creating a throughbore and counterbore. aspects of a tunnel, and various tools for suturing and anchoring a graft in place. These additional tools and instruments are not shown so as not to further complicate the figure.

Typically, a surgical procedure such as an ACL repair starts with imaging (e.g., X-ray imaging, computed tomography (CT), magnetic resonance imaging (MRI)) of the surgical site, including the relevant anatomy of the patient. For an ACL repair, this may include imaging the lower portion of a patient's femur, the upper portion of the patient's tibia, and the articular cartilage. Imaging in this context may involve capture of multiple cross-sectional images or slices. The discussion that follows assumes MRI imaging, but again many different types of imaging may be used. The MRI imaging can be segmented from the image slices such that a volumetric model or three-dimensional model of the anatomy is created. Any suitable currently available, or after developed, segmentation technology may be used to create the three-dimensional model.

Intraoperative aspects include steps and procedures for setting up the surgical system to perform the various repairs. It is noted, however, that some of the intraoperative aspects (e.g., optical system calibration), may take place before any ports or incisions are made through the patient's skin, and in fact before the patient is wheeled into the surgical room. Nevertheless, such steps and procedures may be considered intraoperative as they take place in the surgical setting and with the surgical equipment and instruments used to perform the actual repair.

A procedure such as an ACL reconstruction is conducted arthroscopically and may be computer-assisted in the sense that the surgical controller 418 may be used for arthroscopic navigation within the surgical site. More particularly, in example systems the surgical controller 418 may provide computer-assistance during procedure by tracking location of various objects within the surgical site, such as the location of the bone within the three-dimensional coordinate space of the view of the arthroscope, and location of the various instruments (e.g., the drill wire 424, the aimer 426) within the three- dimensional coordinate space of the view of the arthroscope.

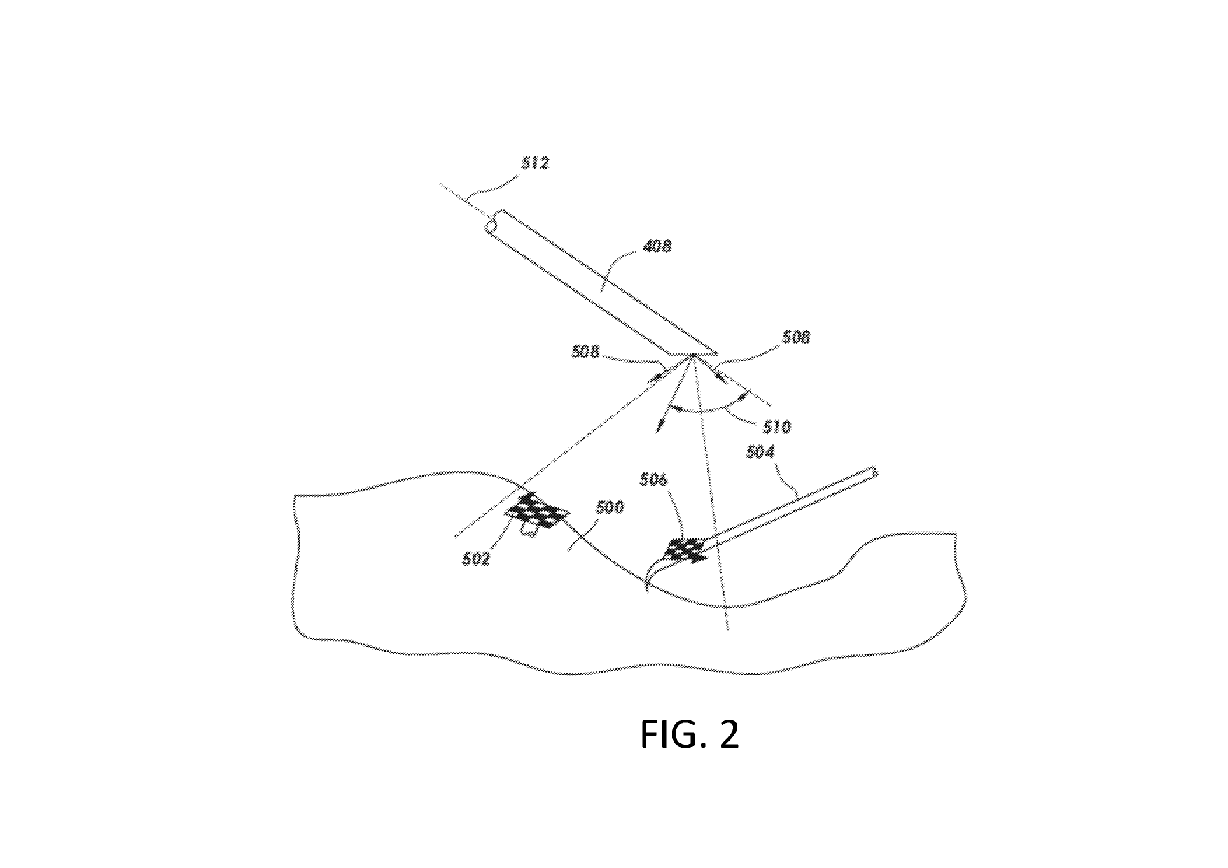

FIG. 2 shows a conceptual drawing of a surgical site with various objects within the surgical site. In particular, visible in FIG. 2 is a distal end of the arthroscope 408, a portion of a bone 500 (e.g., femur), a bone fiducial 502 within the surgical site, a touch probe 504, and a probe fiducial 506. Each is addressed in turn.

The distal end of the arthroscope 408 is designed and constructed to illuminate the surgical site with visible light received by way of the light post 454 (FIG. 1). In the example of FIG. 2, the illumination is illustrated by arrows 508. The illumination provided to the surgical site is reflected by various objects and tissues within the surgical site, and the reflected light that returns to the distal end and enters the arthroscope 408, propagates along an optical channel within the arthroscope 408, and is eventually incident upon a capture array within the camera 410 (FIG. 1). The images detected by the capture array within the camera 410 are sent electronically to the surgical controller 418 (FIG. 1) and displayed on the display device 414 (FIG. 1). The images may typically be regarded as video frames in a stream of video captured by the camera 410. In accordance with example systems, the arthroscope 408 has a single optical path for capturing images of the surgical site, notwithstanding that the single optical path may be constructed of two or more optical members (e.g., glass rods, optical fibers). That is to say, in example systems and methods the computer-assisted navigation provided by the arthroscope 408, camera 410, and surgical controller 418 is provided with the arthroscope 408 that is not a stereoscopic endoscope having two distinct optical paths separated by an interocular distance at the distal end endoscope.

During a surgical procedure, a surgeon selects an arthroscope with a viewing direction beneficial for the planned surgical procedure. Viewing direction refers to a line residing at the center of an angle subtended by the outside edges or peripheral edges of the view of an endoscope. The viewing direction for some arthroscopes is aligned with the longitudinal central axis of the arthroscope, and such arthroscopes are referred to as "zero degree" arthroscopes (e.g., the angle between the viewing direction and the longitudinal central axis of the arthroscope is zero degrees). The viewing direction of other arthroscopes forms a non-zero angle with the longitudinal central axis of the arthroscope. For example, for a 30° arthroscope the viewing direction forms a 30° angle to the longitudinal central axis of the arthroscope, the angle measured as an obtuse angle beyond the distal end of the arthroscope. In many cases for ACL repair, the surgeon selects a 30° arthroscope or a 45° arthroscope based on location of the port created through the skin of the patient. In the example of FIG. 2, the view angle 510 of the arthroscope 408 forms a non-zero angle to the longitudinal central axis 512 of the arthroscope 408.

The arthroscope 408 is rotationally adjustable with respect to the camera 410 about the longitudinal central axis 512 of the arthroscope 408, to enable adjustment of the direction of the field of view of the arthroscope 408 and thus the field of view of the camera 410 via the arthroscope 408. A user of the arthroscopic assembly may adjust the rotational position of the arthroscope 408 about the axis 512 by gripping the light post 454 and turning the light post 454 so that it turns the arthroscope 408 about the axis 512 relative to the camera 410.

Still referring to FIG. 2, within the view of the arthroscope 408 is a portion of the bone 500, along with the bone fiducial 502, the touch probe 504, and the probe fiducial 506. The bone fiducial 502 is shown as a planar element having a pattern disposed thereon, though other shapes for the bone fiducial 502 may be used (e.g., a square block with a pattern on each face of the block). The bone fiducial 502 may be fixed in position with respect to the bone 500 in any suitable form (e.g., a fastener, such as a screw, or otherwise maintained stationary with respect to the bone 500). The bone fiducial 502 is intended, by its physical attachment to the bone or otherwise, to remain in one place for the duration of a procedure, whether or not it is within the field of view of arthroscope 408. The pattern of the bone fiducial is designed to provide information regarding the orientation of the bone fiducial 502 in the three-dimensional coordinate space of the view of the arthroscope 408. More particularly, the pattern is selected such that the orientation of the bone fiducial 502, and thus the orientation of the underlying bone 500, may be determined from images captured by the arthroscope 408 and attached camera 410 (FIG. 1).

The probe fiducial 506 is shown as a planar element attached to the touch probe 504. The touch probe 504 may be used, as discussed more below, to "paint" the surface of the bone 500 as part of the registration of the bone 500 to a three-dimensional bone model, and the touch probe 504 may also be used to indicate - for tunnel reconstruction procedures, for example - revised-tunnel entry locations in the case of intraoperative changes to the tunnel paths. The probe fiducial 506 is shown as a planar element having a pattern disposed thereon, though other shapes for the probe fiducial 506 may be used (e.g., a square block surrounding the touch probe 504 with a pattern on each face of the block). The pattern of the probe fiducial 506 is designed to provide information regarding the orientation of the probe fiducial 506 in the three-dimensional coordinate space of the view of the arthroscope 408. More particularly, the pattern is selected such that the orientation of the probe fiducial 506, and thus the location of the tip of the touch probe 504 (i.e., the tool tip), may be determined from images captured by the arthroscope 408 and attached camera 410 (FIG. 1).

Other instruments within the view of the arthroscope 408 may also have fiducials, such as the drill wire 424 (FIG. 1) and aimer 426 (FIG. 1), but the additional instruments are not shown so as not unduly complicate the figure. Moreover, in addition to or in place of tracking location based on the view through the arthroscope 408, the location of the distal end of one or more of the instruments may be tracked by other methods and systems. For example, for devices that rigidly extend out of the surgical site (e.g., the aimer 426 (FIG. 1)), the location may be tracked by an optical array coupled to the aimer and viewed through the camera 412 (FIG. 1) such as a stereoscopic camera. As another example, the shape of an instrument such as a touch probe may itself be automatically recognizable, for example as a result of programming and/or machine-learning enabling recognition of the instrument in arthroscopic images, such that the position and orientation of the instrument may be gleaned automatically without it having to itself carry a fiducial. The location within the three-dimensional coordinate space of the camera 412 is then transformed into the three-dimensional coordinate space of the view of the example arthroscope to determine location of the distal end within the surgical site.

The images captured by the arthroscope 408 and attached camera 410 are subject to optical distortion in many forms. For example, the visual field between distal end of the arthroscope 408 and the bone 500 within the surgical site is filled with fluid, such as bodily fluids and saline used to distend the joint. Many arthroscopes have one or more lenses at the distal end that widen the field of view, and creating a wider field of view causes a "fish eye" effect in the captured images. Further, the optical elements within the arthroscope (e.g., rod lenses) may have optical aberrations inherent to the manufacturing and/or assembly process. Further still, the camera 410 may have various optical elements for focusing the images receives onto the capture array, and the various optical elements may have aberrations inherent to the manufacturing and/or assembly process. As explained in further detail in Quist, in example systems and methods, prior to use within each surgical procedure, the endoscopic optical system is calibrated to account for the various optical distortions. In an example calibration procedure, the example surgical controller 418 creates a characterization function that characterizes optical distortion between the calibration target and the capture array within the camera 410. The characterization function may include a calibration for determining orientation of fiducial markers visible within the surgical site (e.g., bone fiducial 502, probe fiducial 506) by way of the arthroscope 408 and attached camera 410.

Registration of a bone model(s) to the anatomy is conducted. That is, during the planning stage, imaging (e.g., MRI) of the knee takes place, including the relevant anatomy like the lower portion of the femur, the upper portion of the tibia, and the articular cartilage. The imaging can be segmented such that a volumetric model or three- dimensional model of the anatomy is created from cross-sectional images captured during the imaging.

During the intraoperative repair, the three-dimensional bone models and the cross-sectional images are provided to the surgical controller 418. Again using the example of ACL repair, the three-dimensional bone model of the lower portion of the femur is provided to the surgical controller 418. Thus, the surgical controller 418 receives the three-dimensional bone model, and assuming the arthroscope 408 is inserted into the knee by way of a port through the patient's skin, the surgical controller 418 also receives video images of the femur. In accordance with example methods, the surgical controller 418 may be provided, and thus may receive, the cross-sectional images captured during the imaging from the planning stage.

In order to relate the three-dimensional bone model to the images received by way of the arthroscope 408 and camera 410, the surgical controller 418 registers the three-dimensional bone model to the images of the femur received by way of the arthroscope 408 and camera 410.

In accordance with example methods, a fiducial marker or bone fiducial (e.g., bone fiducial 502 of FIG. 2) is attached or otherwise fixed in place with respect to the femur. The bone fiducial placement is such that the bone fiducial is within the field of view of the arthroscope 408, but in a location spaced apart from a region of interest for the procedure.

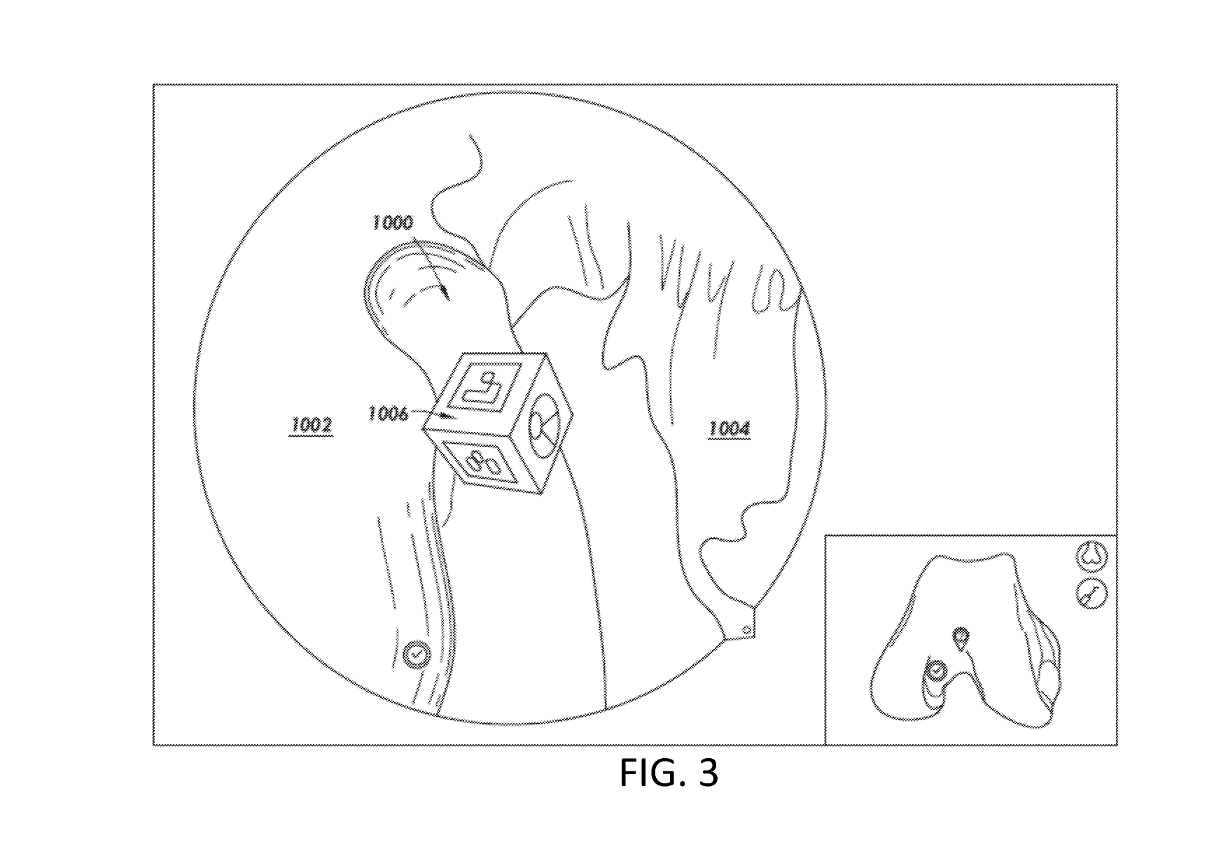

FIG. 3 is an example video display showing portions of a femur and a bone fiducial. The display may be shown, for example, on the display device 414 (FIG. 1) associated with the device cart 402 (FIG. 1), or any other suitable location. In particular, visible in FIG. 3 is a femoral notch or intercondylar notch 1000, a portion of the lateral condyle 1002, a portion of the medial condyle 1004, and an example bone fiducial 1006. The bone fiducial 1006 is a fiducial comprising a cube member. Of the six outer faces of the cube member, the bottom face is associated with an attachment feature (e.g., a screw). The bottom face will be close to or will abut the bone when the bone fiducial 1006 is secured in place, and thus will not be visible in the view of the arthroscope 408 (FIG. 1). The outer face opposite the bottom face includes a placement feature used to hold the bone fiducial 1006 prior to placement, and to attach the bone fiducial 1006 to the underlying bone. Of the remaining four outer faces of the cube member (only two of the remaining faces are visible), each of the four outer faces has a machine-readable pattern thereon, and in some cases each machine-readable pattern is unique. Once placed, the bone fiducial 1006 represents a fixed location on the outer surface of the bone in the view of the arthroscope 408, even as the position of the arthroscope 408 is moved and changed relative to the bone fiducial 1006. Initially, the location of the bone fiducial 1006 with respect to the three-dimensional bone model is not known to the surgical controller 418, hence the need for the registration of the three-dimensional bone model.

In order to relate or register the bone visible in the video images to the three- dimensional bone model, and accordingly to relate or register the bone visible in the video images to respective 3D cylinder models, the surgical controller 418 (FIG. 1) is provided and thus receives a plurality of locations of an outer surface of the bone. For example, the surgeon may touch a plurality of locations using the touch probe 504 (FIG. 2). As previously discussed, the touch probe 504 comprises a probe fiducial 506 (FIG. 2) visible in the video images captured by the arthroscope 408 (FIG. 1) and camera 410 (FIG. 1). The physical relationship between the distal end of the touch probe 504 and the probe fiducial 506 is known by the surgical controller 418, and thus as the surgeon touches each of the plurality of locations on the outer surface of the bone, the surgical controller 418 gains an additional "known" locations of the outer surface of the bone relative to the bone fiducial 1006. Given that the touch probe 504 is a relatively inflexible instrument, in other examples the tracking of the touch probe 504 may be by optical tracking of an optically-reflective array outside the surgical site (e.g., tracking by the camera 412 (FIG. 1)) yet attached to the portion of the touch probe 504 inside the surgical site.

In some cases, particularly when portions of the outer surface of the bone are exposed to view, receiving the plurality of locations of the outer surface of the bone may involve the surgeon "painting" the outer surface of the bone. "Painting" is a term of art that does not involve application of color or pigment, but instead implies motion of the touch probe 504 when the distal end of the touch probe 504 is touching bone.

Further details of registering a three-dimensional bone model to images of a bone received by way of the arthroscope 408 and camera 410 will not be described further herein. However, a number of systems, methods and procedures for conducting such a registration are described in Quist.

Using the three-dimensional bone model and any additional information pertaining to the particular procedure to be conducted, an operative plan may be created. In some cases, however, the surgeon may elect intraoperatively not to adhere strictly to planning. Such an election can be based any of a number of reasons. Regardless of the reason for the election to not adhere strictly to planning, in example systems the surgical controller 418 may enable the surgeon to intraoperatively select alternative approaches as befits the procedure to be conducted.

An example of an alternative approach may be to additionally conduct a measurement, or some other characterization, of a feature that has been observed intraoperatively by the surgeon via an arthroscope. For example, a surgeon may observe a focal defect or a tear, and may wish to make unplanned measurements during observation. It may be useful for a surgeon to use an arthroscopic camera and image processing to conduct such other procedures.

It may also be useful for the surgeon to record measurements, indicate values, or otherwise select options to load applications to handle additional workflows or deviations from a planned workflow. However, it may be useful for the surgeon to be able to record such measurements, indicate such values, or otherwise select options without having to "break scrub" and interact with another device such as a tablet after manipulating an arthroscopic assembly to provide a field of view into a surgical site.

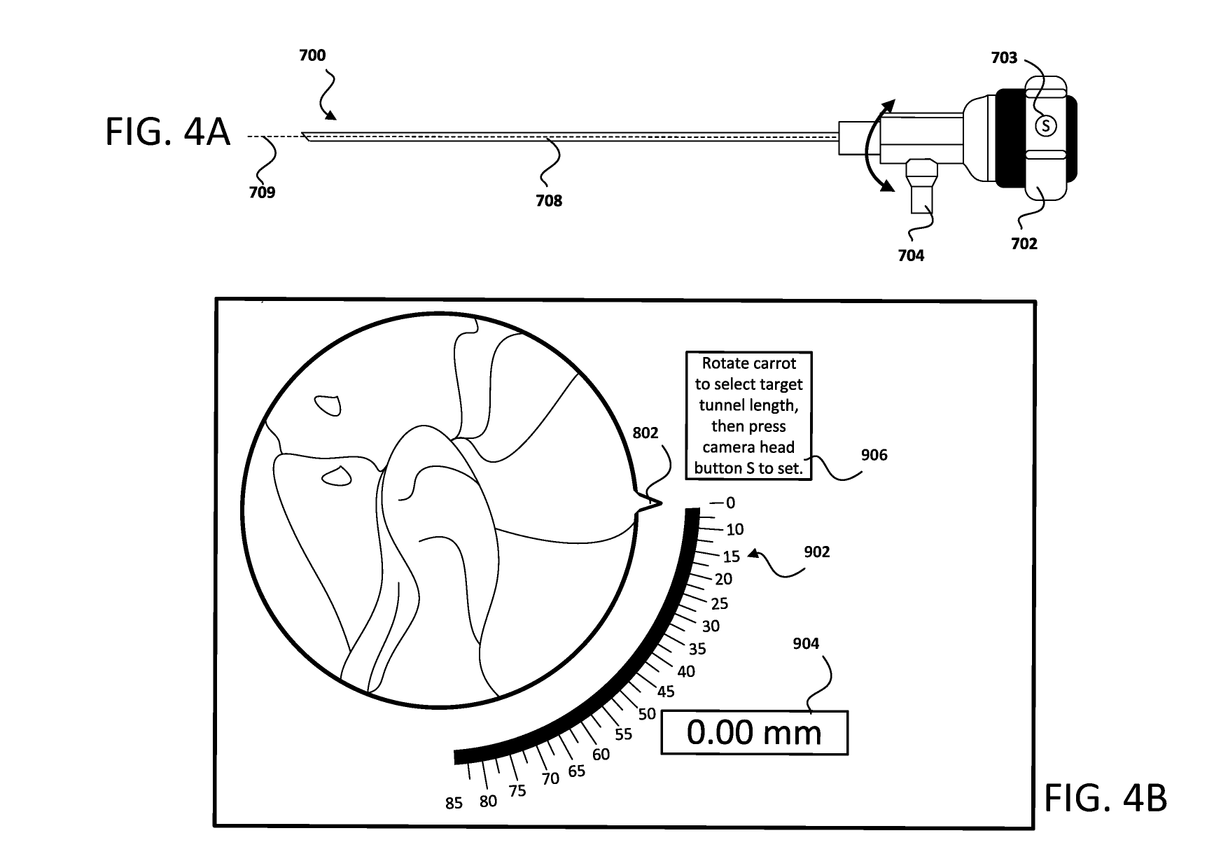

FIG. 4A shows a side view of an arthroscopic assembly 700 with an arthroscope 708 in a first rotational position about its longitudinal central axis 709 with respect to an attached camera 702, in accordance with at least some embodiments. A button 703 is presented on the camera 702 for registering a selection by a user, as will be described. Arthroscopic assembly 700 defines a light connection or light post 704 to which light is provided, and the light is routed internally within the arthroscope 708 to illuminate a surgical field of a surgical site at the distal end of the arthroscope 708

FIG. 4B shows an example video display of a display device showing a video frame received from the camera 702 and caused by the surgical controller 418 to be displayed at a location on the display device. In the video frame, a portion of the surgical site within the field of view of the camera 702 is shown. Also, an indicia 802 that is also within the field of view of the attached camera 702 and in a first location corresponding to the rotational position of the arthroscope 708 in FIG. 4A is shown. Additionally displayed by the surgical controller 418, in association with the video frames, is a field of user options 902. In this example, the field of user options is a range of numbers, with the numbers ranging from 0-85 and representing a millimetre length value. A region 904 displays a current selectable value within the range of numbers corresponding to the first location of the indicia 802. In FIG. 4B, indicia 802 is positioned such that the notional radial line coincides with a location within field of user options 902 that corresponds with the value of 0.00 mm, which is displayed in region 904. Another region 906 displays simple text information, including an instruction for the user to press button 703 on camera 702 in order to select the selectable option in the field of user options 902 corresponding to the position of indicia 802.

In this example, the indicia 802 is shaped as a pointer - generally, a triangle or "carrot," such as a field stop carrot - with a pointing direction that extends radially from the generally circular field of view of the camera 702. As such, indicia 802 is aligned with a notional radial line extending from the center of the field of view of the camera 702 (into the page in FIG. 4B, and generally corresponding to axis 708 in FIG. 4A) that also extends outwards and through the field of user options 902 to thereby also coincide with a corresponding selectable option within the field of user options 902. By adjusting the rotational position of the arthroscope, the position of the indicia 802 tracking the rotational position changes, thus enabling the notional radial line to coincide with a different corresponding selectable option within the field of user options.

In this example, the field of user options 902 itself generally aligns with only one quadrant of the generally circular arthroscopic field of view. Alternatives in which a field of options 902 is larger or smaller are possible, though certain usability constraints and selectability constraints may limit how small or large the field of options 902 for a given application may be. For example, a user such as a surgeon may not wish to rotate the arthroscope 708 very far in order to indicate one of the values in a given field of options 902, as the primary function of the arthroscope 708 is to capture a particular region of interest in the surgical site; turning the arthroscope 708 very far away from the particular region of interest in order just to specify an option may not be desirable. It may be more desirable to arrange the display of the field of options 902 such that only small adjustments of the rotational position of the arthroscope 708 are required to definitely specify an unique option in the field of options 902.

Furthermore, the arthroscope 708 may be in a particular rotational position at the time the surgical system receives a user request to first present a field of user options and to receive input from the user about selecting from the field of user options, with the particular rotational position corresponding to a position of indicia 802 not coinciding with the bottom-right quadrant of the field of view near which field of user options 902 is presented in FIG. 4B. For example, the indicia 802, at the time of the user request to present a field of user options, may be aiming towards the top-left quadrant. Therefore, in examples, surgical system may position a field of user options on the display device based on the current location of the indicia in the video frames when the user request to be presented with the field of user options is received by the surgical system. More particularly, if indicia 802 is aiming towards the top-left quadrant, the surgical system may configure the field of user options to be displayed not in association with the bottom-right quadrant of the field of view, but instead in association with the top-left quadrant. In this way, the indicia 802 may already be directed at a value within the field of user options, and the user may only have to rotate the arthroscope 708, and thus the indicia 802 that tracks the rotational position of the arthroscope in the field of view of camera 702, a small amount in order to indicate and then select a desired option in the field of user options 902. It will be appreciated that the shape of, and orientation on the display device of values, in the field of user options, may be different than that shown in FIG. 4B if the field of user options is to be presented in a different location in association with the field of view of the camera 702.

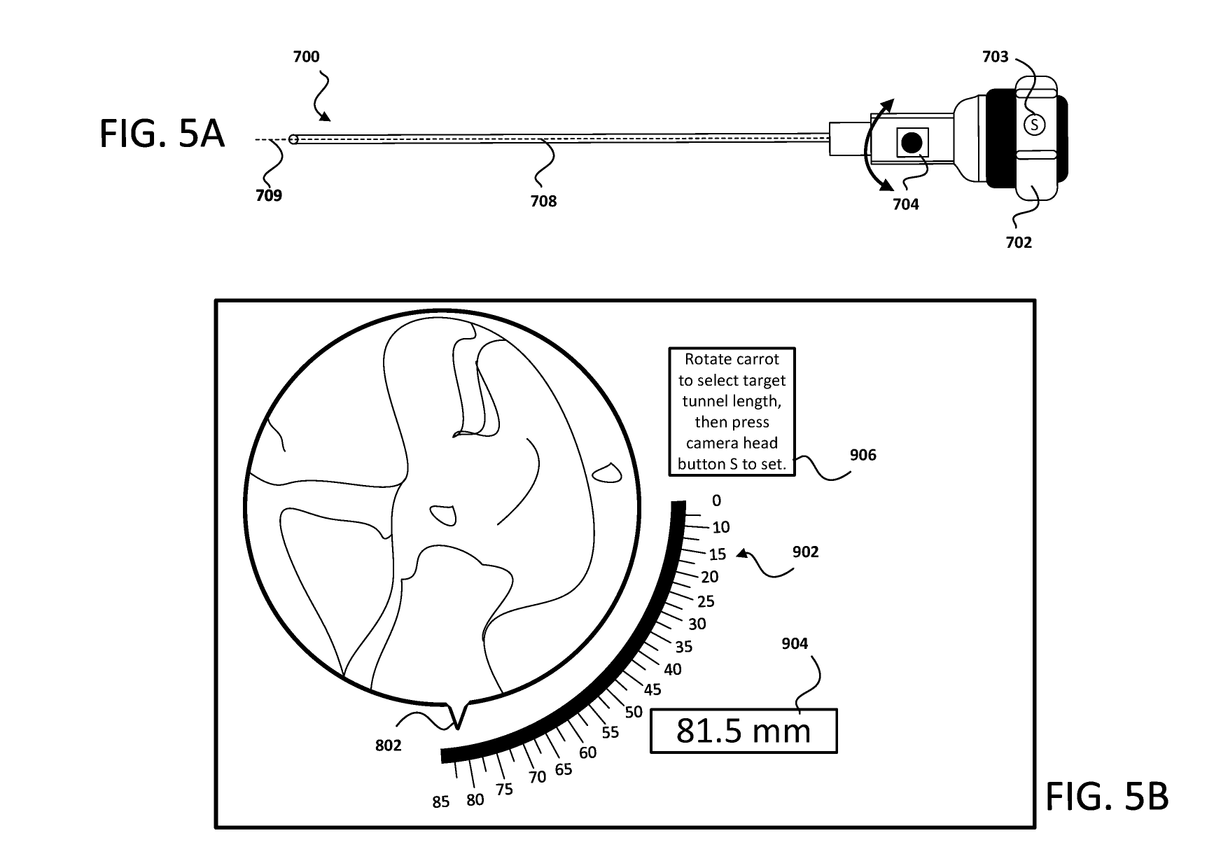

FIG. 5A shows a side view of the arthroscopic assembly 700 with arthroscope 708 in a second rotational position about its longitudinal central axis 709 with respect to attached camera 702, in accordance with at least some embodiments. It may be considered that a user has grasped light post 704 and rotated arthroscope 708 close to ninety (90) degrees clockwise (when looking from camera 702 towards distal end of arthroscope 708) about axis 709 with respect to camera 702 as compared with the rotational position shown in FIGS. 4A and 4B.

FIG. 5B shows the example video display of a display device showing a video frame received from the camera 702. Like in FIG. 4B, in the video frame, a portion of the surgical site within the field of view of the camera 702 is shown. Also, indicia 802 also within the field of view of the attached camera 702 is in a second location corresponding to the rotational position of the arthroscope 708 in FIG. 5A. Additionally displayed, in association with the video frames, is the field of user options 902 - the range of numbers. In this example, the region 904 displays a current selectable value within the range of numbers corresponding to the second location of indicia 802. In FIG. 5B, indicia 802 is positioned such that the notional radial line coincides with a location within field of user options 902 that corresponds with the value of 81.5 mm, which is displayed in region 904. Also, region 906 continues to display simple text information, including the instruction for the user to press button 703 on camera 702 in order to select the selectable option in the field of user options 902 corresponding to the position of indicia 802.

The user may make use of the user interface offered by the indicia 802 and the field of user options 902 to record measurements or to otherwise select options or values, without having to "break scrub" by interacting with a tablet or other data entry device, and instead by simply using the devices the user is already handling.

It will be noted that, in this example, the range of numbers of the field of user options 902 displays numbers only in increments of 5mm. In FIG. 5B, indicia 802 is positioned to indicate, in field of user options 902, a value between 80mm and 85mm. In this example, rather than snapping the value at one of 80mm and 85mm, the surgical system calculates the intermediate value - a number that is between the particular displayed numbers - based on the relative position indicated between the displayed values by the position of indicia 802. Alternatives are possible. For example, it may be useful to snap the value to be selected at one of the displayed values, rather than at an intermediate value, such as for example if the displayed options are menu options or it is desirable to have only integer values selected for a particular implementation or application.

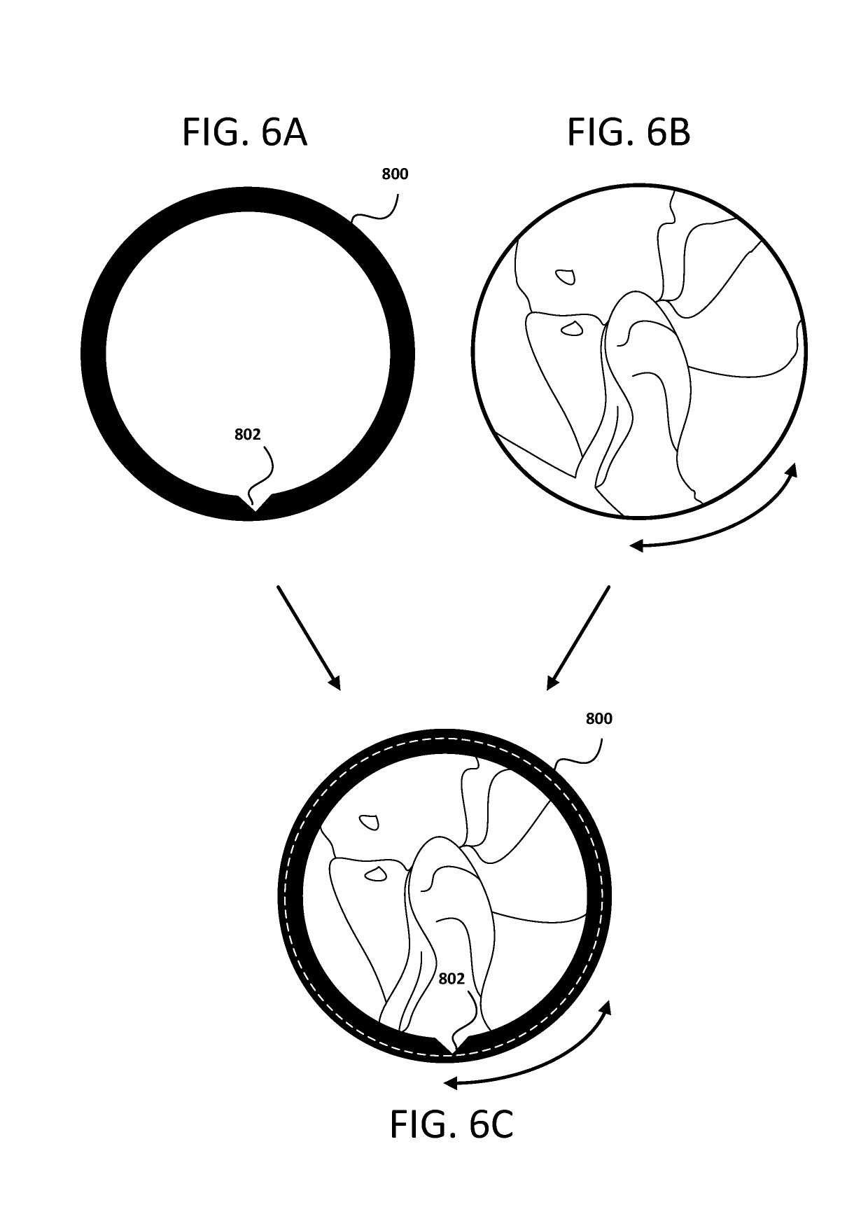

FIG. 6A shows a mask 800 that can be applied to the arthroscope 708 to provide an indicia 802 within the field of view of the attached camera 702 that can track, in the field of view of the camera 702, the rotational position of the arthroscope 708 about the axis 709. FIG. 6B shows a field of view of the camera 702 without the mask 800, and FIG. 6C shows the field of view of the camera 702 including the mask 800, with the mask 800 having been applied to the arthroscope 708 to provide the tracking indicia 802 that is within the field of view of the camera 702 when attached to the arthroscope 708. It will be appreciated that other ways of associating an indicia 802 with arthroscope 708 to track the rotational position of the arthroscope 708 in the field of view of camera 702 are possible. For example, instead of using a mask 800, an unique indicia 802 could be etched or otherwise integrated within the optical path of arthroscope 708. It will be appreciated that the indicia 802 has to be easily, and preferably efficiently, distinguishable in the field of view of camera 702 from other objects - such as objects within the surgical site itself - that are also within the field of view of camera 702.

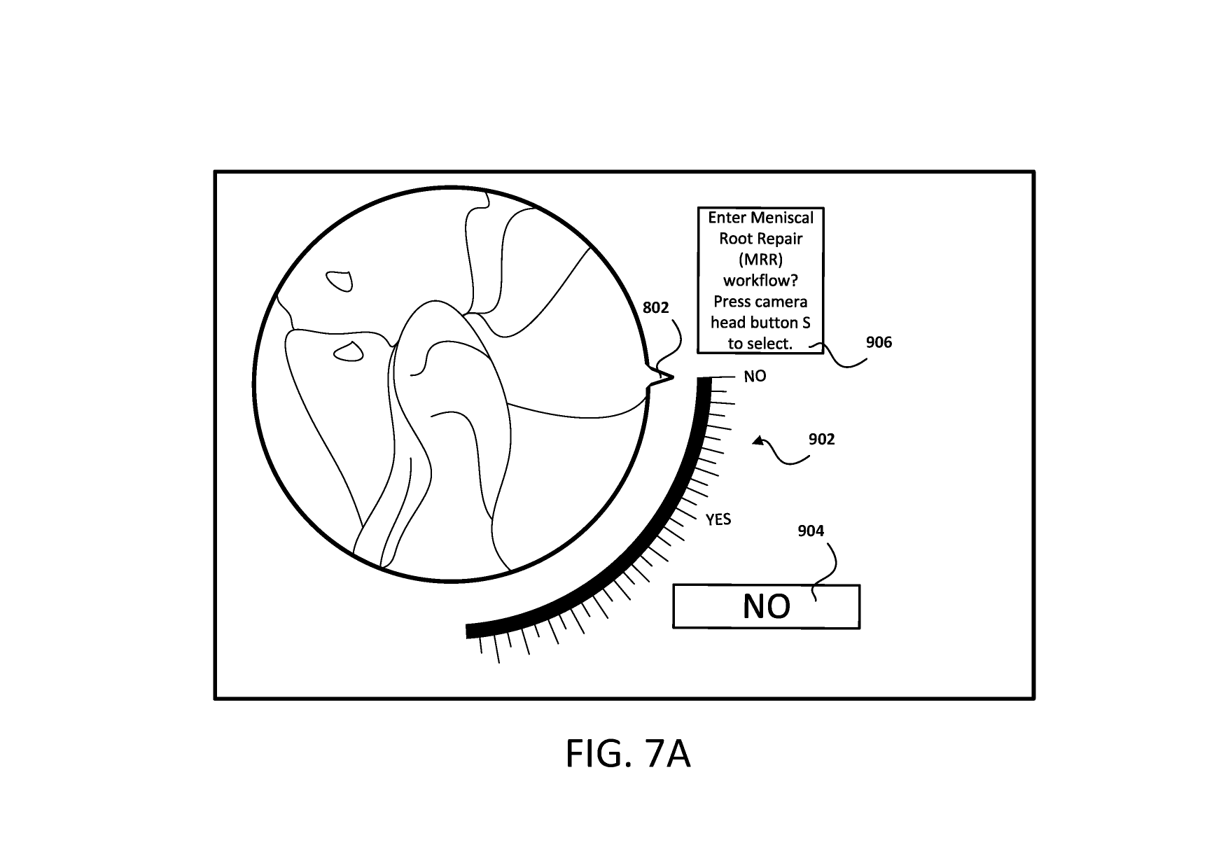

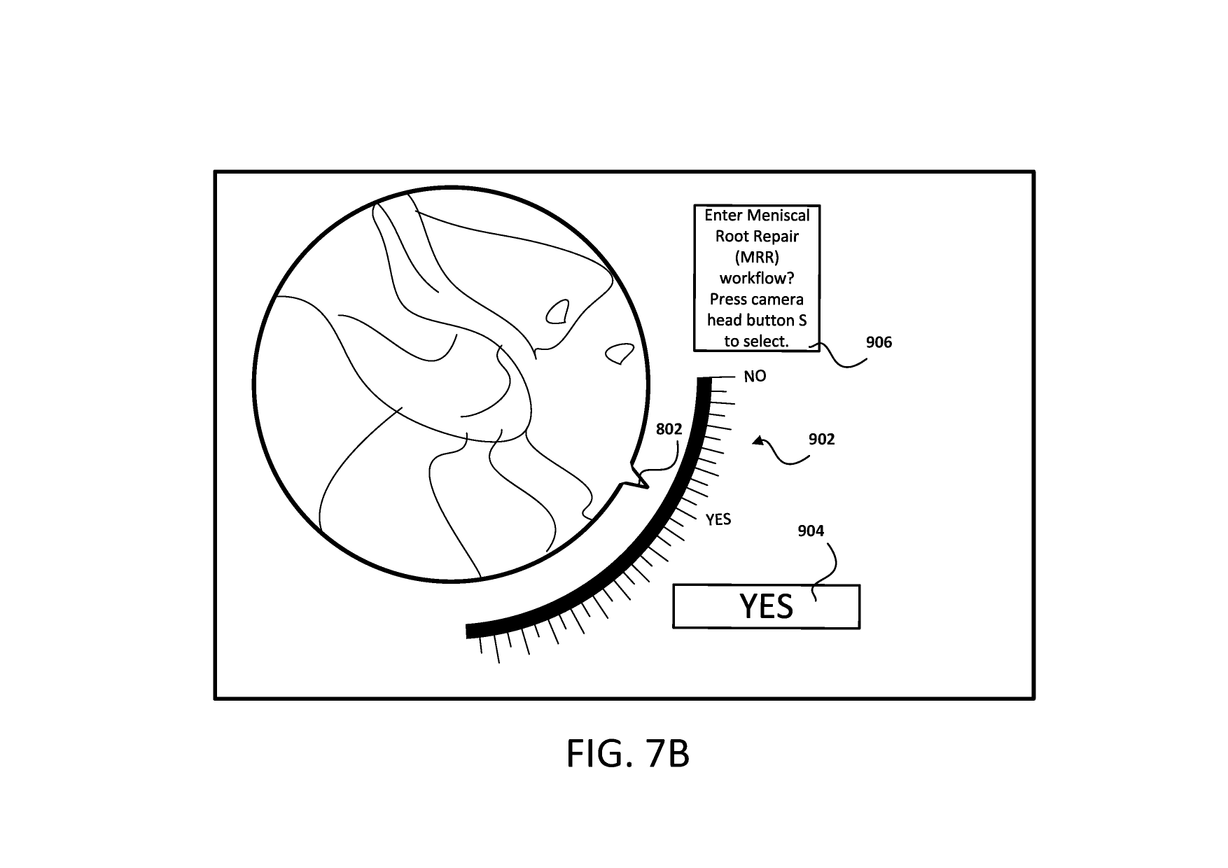

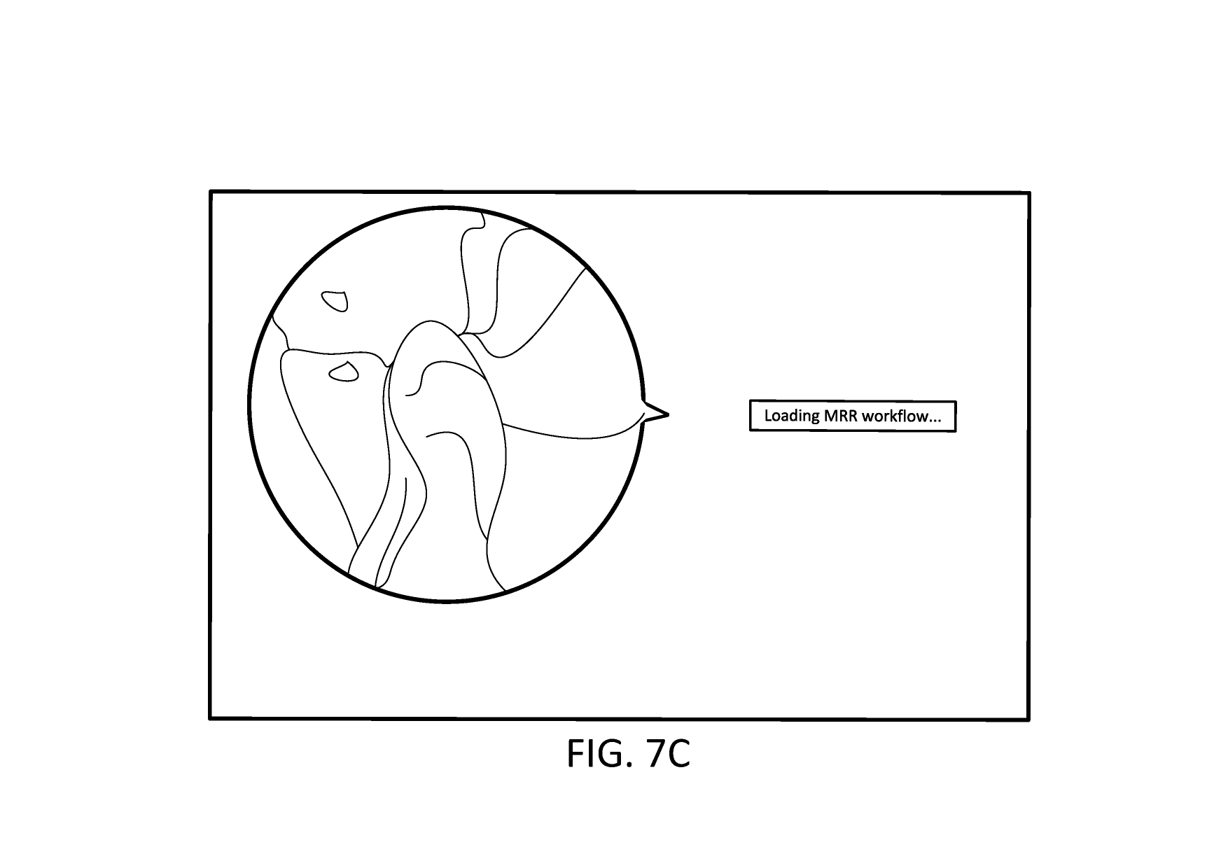

FIG. 7A shows an example video display showing a first location of an indicia 802 in the field of view of an attached camera 702 corresponding to the current rotational position of the arthroscope 708, and showing a menu of two selectable options ("NO", and "YES") and a current one of the selectable options ("the "NO" option) corresponding to a current location of the indicia 802 being displayed in region 904.

FIG. 7B shows the example video display of FIG. 7A in which Indica 802 is in a second location in the field of view of camera 702 corresponding to the current rotational position of arthroscope 708 as adjusted compared to FIG. 7A,and showing the menu of the two selectable options("NO" and " YES") and a current one of the selectable options (the "YES"option) corresponding to a current location of the indicia being displayed in region 904.

FIG. 7C shows the example video display of FIGS. 7A and 7B after a user input signal to select a current selectable option (in this case, the "YES" option) has been received by the surgical system. In this example, the user has pressed button 703 on camera 702 in order to select the "YES" option, and the surgical system may thereafter conduct processing using the selected option, in this example to load an MRI workflow application.

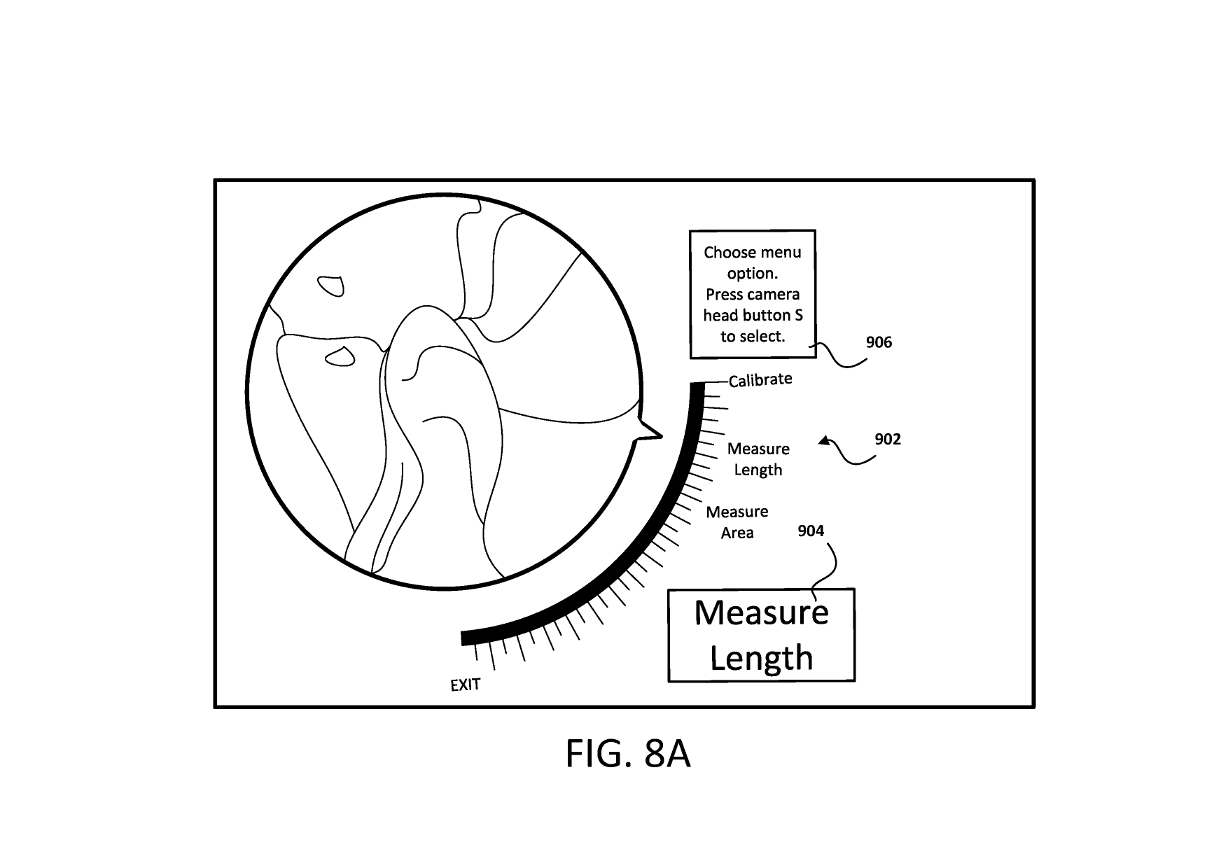

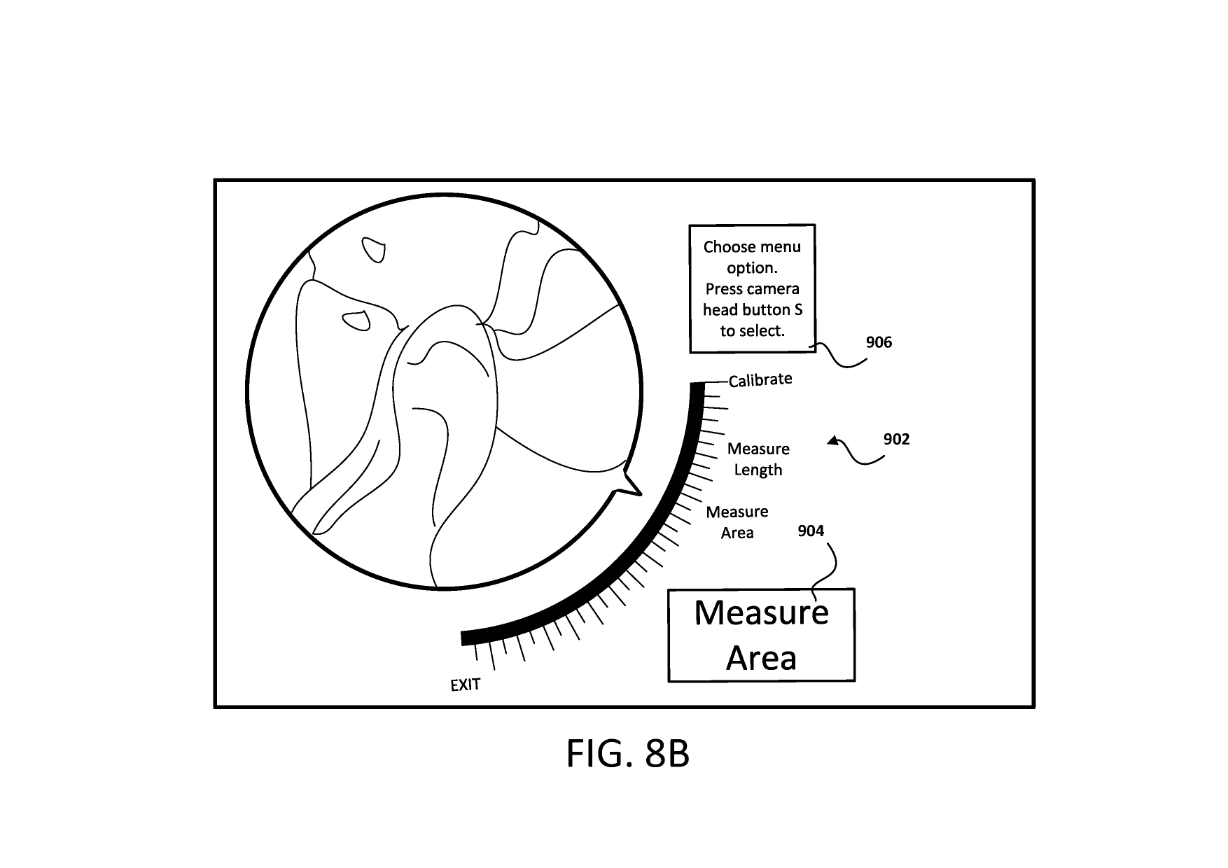

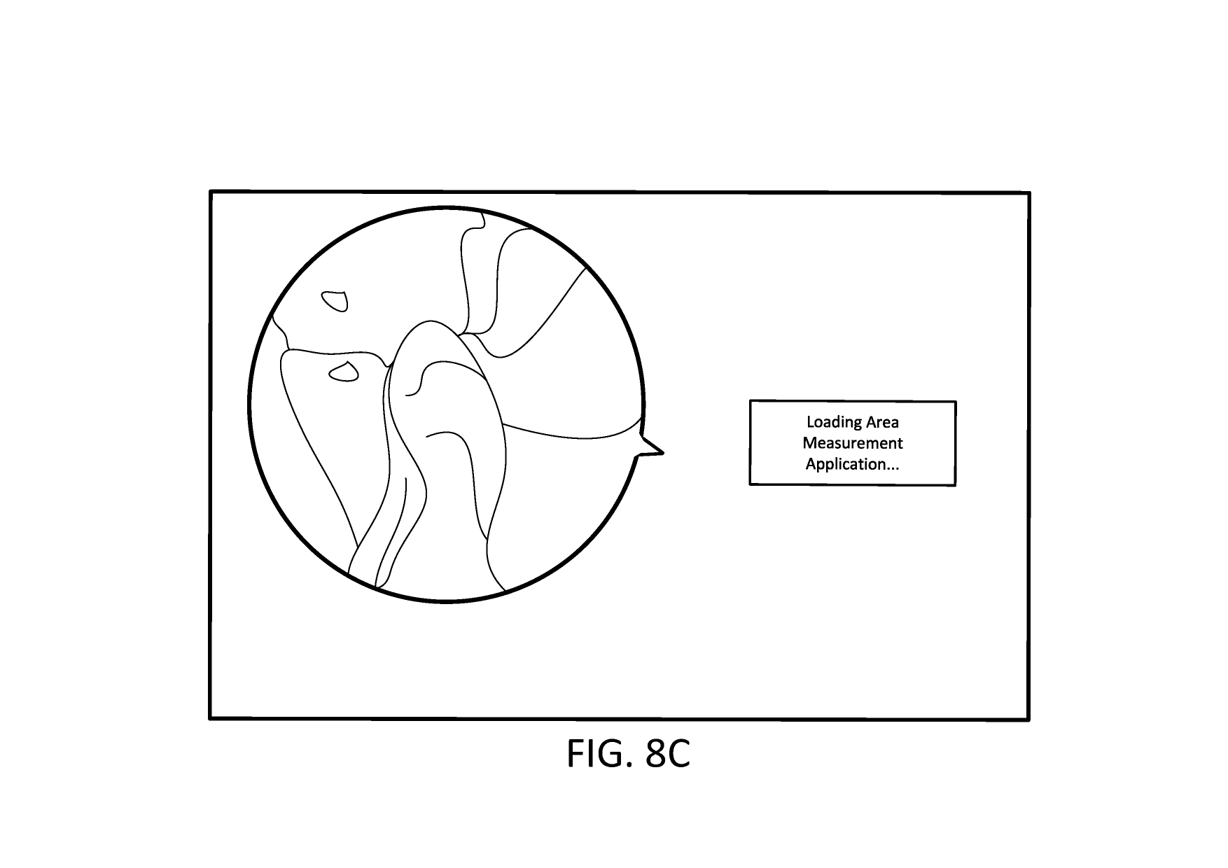

FIG. 8A shows an example video display showing a first location of an indicia 802 in the field of view of an attached camera 702 corresponding to the current rotational position of the arthroscope 708, and showing a menu of four selectable options ("Calibrate", "Measure Length", "Measure Area", and "Exit") and a current one of the selectable options ("the "Measure Length" option) corresponding to a current location of the indicia 802 being displayed in region 904.

FIG. 8B shows the example video display of FIG. 8A in which indicia 802 is in a second location in the field of view of camera 702 corresponding to the current rotational position of arthroscope 708 as adjusted compared to FIG. 8A, and showing the menu of the four selectable options ("Calibrate", "Measure Length", "Measure Area", and "Exit") and a current one of the selectable options (the "Measure Area" option) corresponding to a current location of the indicia being displayed in region 904.

FIG. 8C shows the example video display of FIGS. 8A and 8B after a user input signal to select a current selectable option (in this case, the "Measure Area" option) has been received by the surgical system. In this example, the user has pressed button 703 on camera 702 in order to select the "Measure Area" option, and the surgical system. may thereafter conduct processing using the selected option, in this example to load an area measurement application.

### SOFTWARE AND HARDWARE

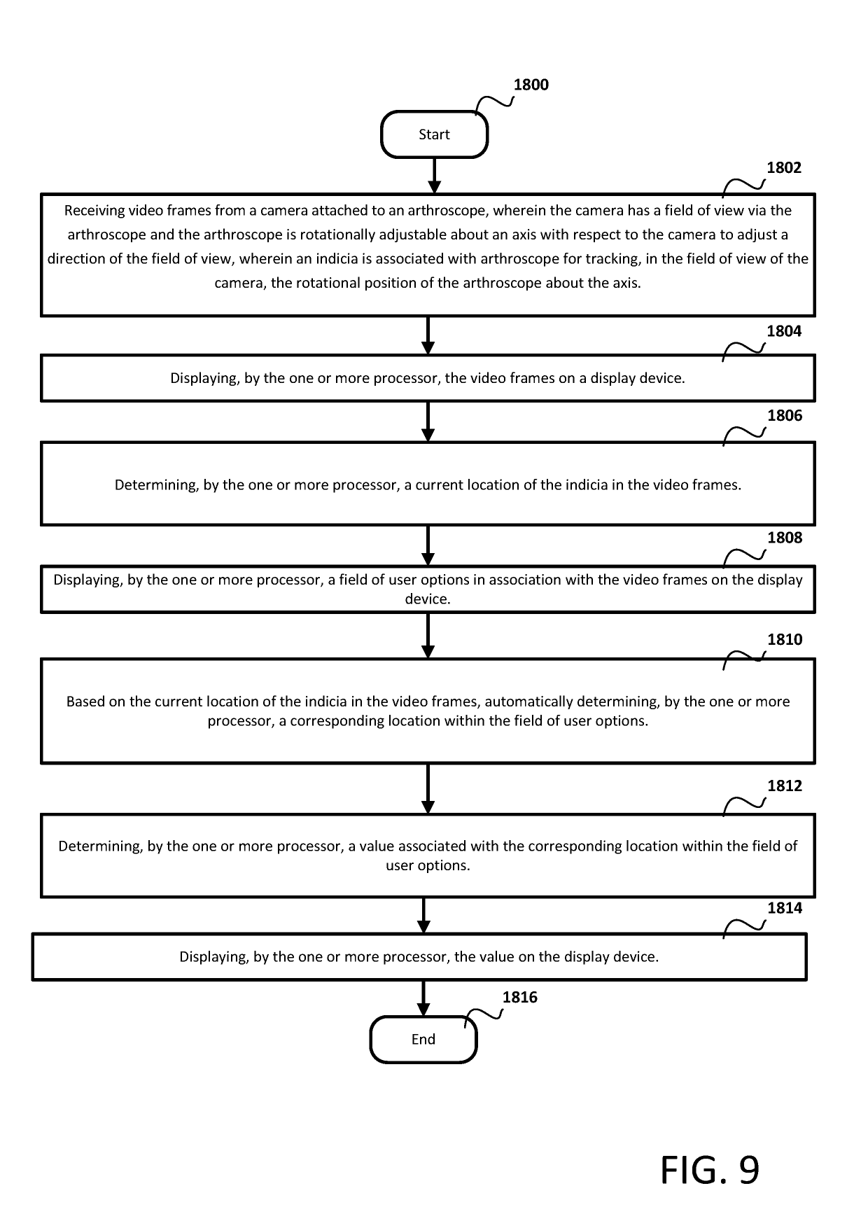

FIG. 9 shows a method, in accordance with at least some embodiments. In particular, the method starts (block 1800) and comprises: receiving, by one or more processor, video frames from a camera attached to an arthroscope, wherein the camera has a field of view via the arthroscope and the arthroscope is rotationally adjustable about an axis with respect to the camera, wherein an indicia is associated with arthroscope for tracking, in the field of view of the camera, the rotational position of the arthroscope about the axis (block 1802), displaying, by the one or more processor, the video frames on a display device (block 1804). The method comprises, during displaying the video frames on the display device: determining, by the one or more processor, a current location of the indicia in the video frames (block 1806); displaying, by the one or more processor, a field of user options in association with the video frames on the display device (block 1808); based on the current location of the indicia in the video frames, automatically determining, by the one or more processor, a corresponding location within the field of user options (block 1810); determining, by the one or more processor, a value associated with the corresponding location within the field of user options (block 1812); and displaying, by the one or more processor, the value on the display device (block 1814). Thereafter, the method ends (block 1816).

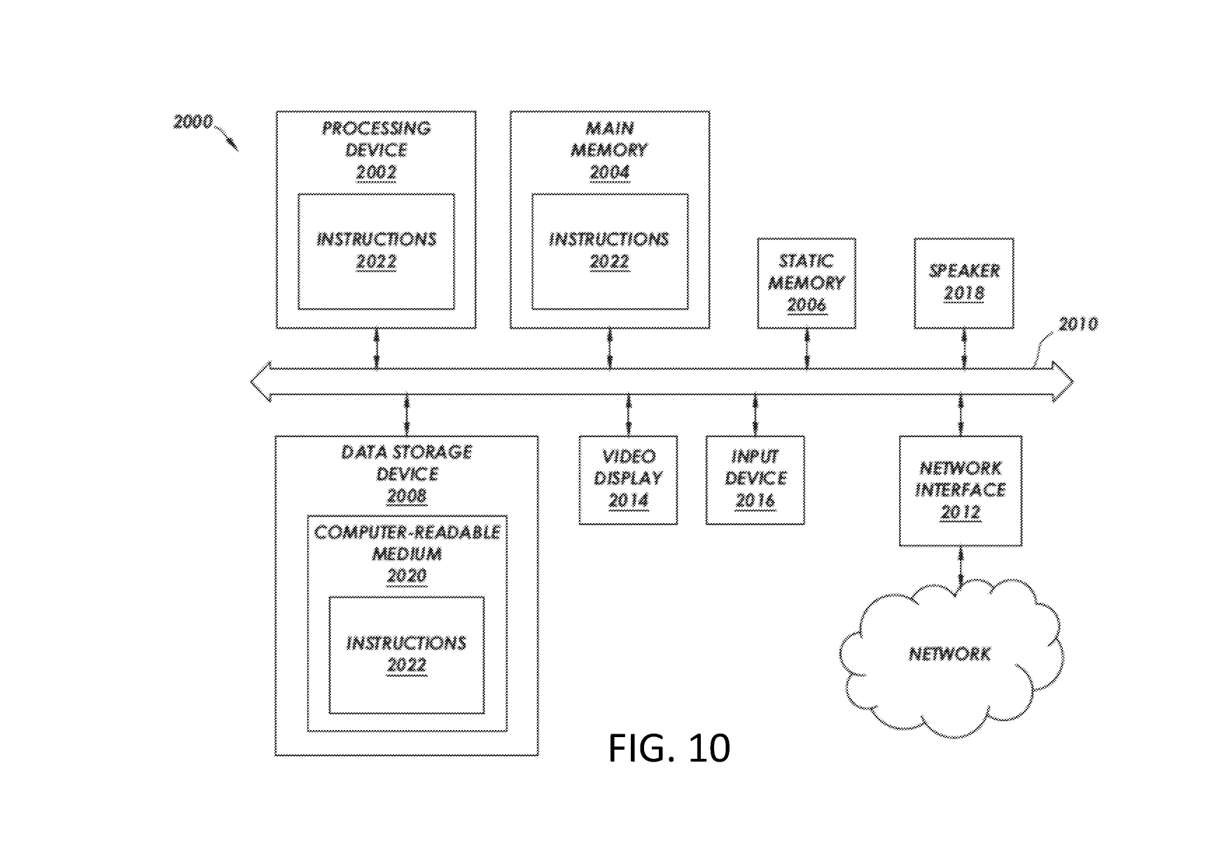

FIG. 10 shows an example computer system 2000. In one example, computer system 2000 may correspond to the surgical controller 418, a tablet device within the surgical room, or any other system that implements any or all the various methods discussed in this specification. The computer system 2000 may be connected (e.g., networked) to other computer systems in a local-area network (LAN), an intranet, and/or an extranet (e.g., device cart 402 network), or at certain times the Internet (e.g., when not in use in a surgical procedure). The computer system 2000 may be a server, a personal computer (PC), a tablet computer or any device capable of executing a set of instructions (sequential or otherwise) that specify actions to be taken by that device. Further, while only a single computer system is illustrated, the term "computer" shall also be taken to include any collection of computers that individually or jointly execute a set (or multiple sets) of instructions to perform any one or more of the methods discussed herein.

The computer system 2000 includes a processing device 2002, a main memory 2004 (e.g., read-only memory (ROM), flash memory, dynamic random access memory (DRAM) such as synchronous DRAM (SDRAM)), a static memory 2006 (e.g., flash memory, static random access memory (SRAM)), and a data storage device 2008, which communicate with each other via a bus 2010.

Processing device 2002 represents one or more general-purpose processing devices such as a microprocessor, central processing unit, or the like. More particularly, the processing device 2002 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 2002 may also be one or more special-purpose processing devices such as an application specific integrated circuit (ASIC), a field programmable gate array (FPGA), a digital signal processor (DSP), network processor, or the like. The processing device 2002 is configured to execute instructions for performing any of the operations and steps discussed herein. Once programmed with specific instructions, the processing device 2002, and thus the entire computer system 2000, becomes a special-purpose device, such as the surgical controller 418.

The computer system 2000 may further include a network interface device 2012 for communicating with any suitable network (e.g., the device cart 402 network). The computer system 2000 also may include a video display 2014 (e.g., display device 414), one or more input devices 2016 (e.g., a microphone, a keyboard, and/or a mouse), and one or more speakers 2018. In one illustrative example, the video display 2014 and the input device(s) 2016 may be combined into a single component or device (e.g., an LCD touch screen).

The data storage device 2008 may include a computer-readable storage medium 2020 on which the instructions 2022 (e.g., implementing any methods and any functions performed by any device and/or component depicted described herein) embodying any one or more of the methodologies or functions described herein is stored. The instructions 2022 may also reside, completely or at least partially, within the main memory 2004 and/or within the processing device 2002 during execution thereof by the computer system 2000. As such, the main memory 2004 and the processing device 2002 also constitute computer-readable media. In certain cases, the instructions 2022 may further be transmitted or received over a network via the network interface device 2012.

While the computer-readable storage medium 2020 is shown in the illustrative examples to be a single medium, the term "computer-readable storage medium" should be taken to include a single medium or multiple media (e.g., a centralized or distributed database, and/or associated caches and servers) that store the one or more sets of instructions. The term "computer-readable storage medium" shall also be taken to include any medium that is capable of storing, encoding or carrying a set of instructions for execution by the machine and that cause the machine to perform any one or more of the methodologies of the present disclosure. The term "computer-readable storage medium" shall accordingly be taken to include, but not be limited to, solid-state memories, optical media, and magnetic media.

While examples have been described, variations are possible.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A surgical system comprising:
an arthroscope;
a camera attached to the arthroscope and having a field of view via the arthroscope, wherein the arthroscope is rotationally adjustable about an axis with respect to the camera;
an indicia associated with the arthroscope for tracking, in the field of view of the camera, the rotational position of the arthroscope about the axis;
a display device; and
one or more processor for:
receiving video frames from the camera;
displaying the video frames on the display device; and
during displaying the video frames on the display device:
determining a current location of the indicia in the video frames;
displaying, on the display device, a field of user options in association with the video frames;
based on the current location of the indicia in the video frames, automatically determining a corresponding location within the field of user options;
determining a value associated with the corresponding location within the field of user options; and
displaying the value on the display device.

2. The surgical system of claim 1, wherein the field of user options is a range of numbers.

3. The surgical system of claim 2, wherein the corresponding location within the range of numbers is a particular number within the range of numbers, and further wherein determining the value associated with the corresponding location comprises determining the value to be the particular number.

4. The surgical system of claim 2, wherein the corresponding location within the range of numbers is a location between two particular numbers within the range of numbers, and further wherein determining the value associated with the corresponding location comprises determining the value to be a number that is between the two particular numbers.

5. The surgical system of claim 1, wherein the field of user options is a set of menu options.

6. The surgical system of claim 5, wherein the set of menu options comprises two menu options.

7. The surgical system of claim 1, wherein the one or more processor is for:
receiving a user input signal to select the determined value; and
conducting processing using the determined value.

8. The surgical system of claim 7, wherein conducting processing using the determined value comprises storing the determined value.

9. The surgical system of claim 1, wherein the field of user options is displayed on the display device responsive to receipt, by the one or more processor, of a user request.

10. The surgical system of claim 9, wherein a position on the display device at which the field of user options is displayed is based on the current location of the indicia in the video frames at the time of the receiving of the user request.

11. A processor-implemented method comprising:
receiving, by one or more processor, video frames from a camera attached to an arthroscope, wherein the camera has a field of view via the arthroscope and the arthroscope is rotationally adjustable about an axis with respect to the camera, wherein an indicia is associated with arthroscope for tracking, in the field of view of the camera, the rotational position of the arthroscope about the axis;
displaying, by the one or more processor, the video frames on a display device;
during displaying the video frames on the display device:
determining, by the one or more processor, a current location of the indicia in the video frames;
displaying, by the one or more processor, a field of user options in association with the video frames on the display device;
based on the current location of the indicia in the video frames, automatically determining, by the one or more processor, a corresponding location within the field of user options;
determining, by the one or more processor, a value associated with the corresponding location within the field of user options; and
displaying, by the one or more processor, the value on the display device.

12. The method of claim 11, wherein the field of user options is a range of numbers.

13. The method of claim 12, wherein the corresponding location within the range of numbers is a particular number within the range of numbers, and further wherein determining the value associated with the corresponding location comprises determining the value to be the particular number.

14. The method of claim 12, wherein the corresponding location within the range of numbers is a location between two particular numbers within the range of numbers, and further wherein determining the value associated with the corresponding location comprises determining the value to be a number that is between the two particular numbers.

15. The method of claim 11, wherein the field of user options is a set of menu options.

16. The method of claim 14, wherein the set of menu options comprises two menu options.

17. The method of claim 11, further comprising:
receiving, by the one or more processor, a user input signal to select the determined value; and
conducting processing, by the one or more processor, using the determined value.

18. The method of claim 17, wherein conducting processing using the determined value comprises storing the determined value.

19. The method of claim 11, wherein the field of user options is displayed on the display device responsive to receiving, by the one or more processor, a user request.

20. The method of claim 19, wherein a position on the display device at which the field of user options is displayed is based on the current location of the indicia in the video frames at a time of the receiving of the user request.
